(19) United States
(12) Patent Application Publication
(10) Pub. No.: US 2023/0110966 A1
(43) Pub. Date: Apr. 13, 2023

(54) METHOD OF IDENTIFYING MICROORGANISMS OF A MICROBIOME

(71) Applicants: Hudson Institute of Medical Research, Clayton, Victoria (AU); Monash University, Clayton, Victoria (AU); Monash Health, Clayton, Victoria (AU)

(72) Inventors: Samuel Charles FORSTER, Bittern, Victoria (AU); Edward Mark GILES, Alphington, Victoria (AU); Gemma Louise D'ADAMO, Bellarine, Victoria (AU)

(21) Appl. No.: 17/904,499

(22) PCT Filed: Feb. 18, 2021
(86) PCT No.: PCT/AU2021/050138

§ 371 Date: Aug. 18, 2022

(30) Foreign Application Priority Data
Feb. 18, 2020 (AU) 2020900450

(51) Int. Cl. C12Q 1/689; C12Q 1/6869

(52) U.S. Cl. CPC C12Q 1/689; C12Q 1/6869

(57) ABSTRACT

The present invention generally relates to methods of identifying microorganisms of the microbiome.

### FIELD OF THE INVENTION

The present invention generally relates to methods of identifying microorganisms of the microbiome.

### BACKGROUND OF THE INVENTION

A microbiome is an ecological community of commensal, symbiotic, and pathogenic microorganisms that are associated with an organism. The human microbiome comprises more microbial cells than human cells, but characterization of the human microbiome is still in nascent stages due to limitations in sample processing techniques, genetic analysis techniques, and resources for processing large amounts of data. Nonetheless, the microbiome is suspected to play at least a partial role in a number of health/disease-related states (e.g., preparation for childbirth, diabetes, autoimmune disorders, gastrointestinal disorders, rheumatoid disorders, neurological disorders, etc.).

Given the profound implications of the microbiome in affecting a subject's health, efforts related to the characterization of the microbiome should be pursued. Current methods and systems for analyzing the microbiomes of an organism, such as in defined regions of a human, have limitations including a large amount of host nucleic acid contamination.

### SUMMARY OF THE INVENTION

The present inventors have developed a new and efficient method for analysing microorganisms of the microbiome of a region of a subject which relies on minimizing host nucleic acid contamination.

In a first aspect, the present invention provides a method of identifying microorganisms of the microbiome of a region of a subject, the method comprising;

i) obtaining a metagenomic sample derived from the region depleted of nucleic acids from the subject,

ii) conducting metagenomic sequencing of nucleic acids in the depleted metagenomic sample from step i), and

iii) analysing the results of the metagenomic sequencing to identify microorganisms present in the microbiome in the region of the subject.

In an embodiment, step i) comprises one or more or all of:

1) culturing in vitro microorganisms from a sample of the microbiome from the region of the subject,

2) hybridizing a probe to DNA of the subject in the metagenomic sample, and depleting the sample of DNA bound to the probe, and

3) hybridizing a probe to DNA of microorganisms expected to be present in the metagenomic sample, and selecting DNA bound to the probe.

In a further embodiment, step i) comprises

a) culturing in vitro microorganisms from a sample of the microbiome from the region of the subject, and

b) obtaining a metagenomic sample from the cultured microorganisms.

In another aspect, the present invention provides a method of identifying microorganisms of the microbiome of a region of a subject, the method comprising;

i) culturing in vitro microorganisms from a sample of the microbiome from the region of the subject,

ii) obtaining a metagenomic sample from the cultured microorganisms,

iii) conducting metagenomic sequencing of nucleic acids in the metagenomic sample from step ii), and

iv) analysing the results of the metagenomic sequencing to identify microorganisms present in the microbiome in the region of the subject.

In an embodiment, the sample is cultured under aerobic conditions. In an alternative embodiment, the sample is cultured under anaerobic conditions. In a further alternative embodiment, the sample is cultured under microaerophilic conditions.

The culturing can be performed on any suitable media. In an embodiment, the microorganisms are cultured on yeast-extract-casitone-fatty acid (YCFA) agar.

In an embodiment, at least one pre-selected region of the DNA is sequenced. In an embodiment, the sequence of 16S ribosomal genes are sequenced or a portion thereof.

In an embodiment, the subject is an animal or a plant. In an embodiment, the animal is a mammal. In an embodiment, the mammal is a human.

The region may be selected from, but not limited to, a region of the gastrointestinal system, the respiratory system, the female reproductive system, the bladder or the skin.

In an embodiment, the region of the gastrointestinal system is a region within the stomach, small intestine, large intestine, caecum or rectum. In an embodiment, the region is the terminal ileum of the small intestine.

In an embodiment, the region of the respiratory system is a region within the lung.

In an embodiment, the region of the female reproductive system is the vaginal region.

In an embodiment, the sample is from a region of the subject with a phenotype of interest. In an embodiment, the phenotype of interest is a diseased state such as the region is inflamed.

In an embodiment, the microorganisms of the microbiome comprise bacteria, fungus, protozoa, viruses, or any combination thereof.

In an embodiment, the microorganisms of the microbiome at least comprise bacteria.

In an embodiment, the viruses include bacteriophages.

In a further embodiment, step iv) comprises comparing the sequences identified in step iii) to a database comprising microbial sequences.

The methods of the invention can be used in studies, such as case/control studies, to identify microorganism which may be associated with a phenotype of interest. Thus, in another aspect the present invention provides a method of identifying a microorganism which may be associated with a phenotype of interest, the method comprising

i) performing the method of the invention, wherein the sample is from a region of the subject with a phenotype of interest,

ii) comparing the microorganisms identified in step i) with those present in the same region of a subject that does not have the phenotype of interest,

wherein microorganisms identified in step i), but which are not present at the same level in the same region of a subject that does not have the phenotype of interest, may be associated with the phenotype of interest.

The present invention can also be used to identify live microorganisms present in a food, drink or probiotic composition. In particular, the method enables the enrichment of nucleic acids from live microorganisms when compared to nucleic acids in the food, drink or probiotic composition from sources such as the biological material from which the food or drink is made, or from dead microorganisms in the food, drink or probiotic composition.

Thus, in one aspect the present invention provides a method of identifying live microorganisms present in a food, drink or probiotic composition, the method comprising;

i) obtaining a metagenomic sample derived from the food, drink or probiotic composition depleted of nucleic acids from a source other than the live microorganisms,

ii) conducting metagenomic sequencing of nucleic acids in the depleted metagenomic sample from step i), and

iii) analysing the results of the metagenomic sequencing to identify live microorganisms present in the food, drink or probiotic composition.

In one embodiment, step i) comprises one or more or all of:

1) culturing in vitro microorganisms from the food, drink or probiotic composition,

2) hybridizing a probe to DNA of the subject in the metagenomic sample, and depleting the sample of DNA bound to the probe, and

3) hybridizing a probe to DNA of microorganisms expected to be present in the metagenomic sample, and selecting DNA bound to the probe.

In another embodiment, step i) comprises

a) culturing in vitro microorganisms from the food, drink or probiotic composition, and

b) obtaining a metagenomic sample from the cultured microorganisms.

In another aspect, the present invention provides a method of identifying live microorganisms present in a food, drink or probiotic composition, the method comprising;

i) culturing in vitro microorganisms from the food, drink or probiotic composition,

ii) obtaining a metagenomic sample from the cultured microorganisms,

iii) conducting metagenomic sequencing of nucleic acids in the metagenomic sample from step ii), and

iv) analysing the results of the metagenomic sequencing to identify live microorganisms present in the food, drink or probiotic composition.

In an embodiment, the method of the above two aspects can be used to determine the viability of probiotic microorganisms in the food, drink or probiotic composition.

In an embodiment, the method of the above two aspects is used to determine which microorganisms have survived, and their relative abundance, after a period of storage of the food, drink or probiotic composition.

In an embodiment, the method of the above two aspects can be used to detect spoilage or contamination of the food, drink or probiotic composition with a microorganism.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Schematic of sample preparations. The different sample preparations for bacterial culturing. Samples from the three bowel regions (Terminal Ileum, Caecum and Rectum) were cultured in three different environments (Aerobic, Anaerobic and Microaerophilic), with two different initial dilution factors ( 1/10 and 1/100).

FIG. 2. Colony forming unit counts (CFUs) obtained from the terminal ileal samples. CFUs obtained from the terminal ileal samples cultured aerobically (A), anaerobically (B) and microaerophilically (C). Mann-Whitney U tests were used to assess statistical significance among the groups of interest, with the following significance cut-offs: p<0.05*, p<0.01**, p<0.001***. n=15 independent patients were included for these analyses.

FIG. 3. Colony forming unit counts (CFUs) obtained from the caecal samples. Colony forming unit counts (CFUs) obtained from the caecal samples cultured aerobically (A), anaerobically (B) and microaerophilically (C Mann-Whitney U tests were used to assess statistical significance among the groups of interest, with the following significance cut-offs: p<0.05*, p<0.01**, p<0.001***. n=15 independent patients were included for these analyses.

FIG. 4. Colony forming unit counts (CFUs) obtained from the rectal samples. Colony forming unit counts (CFUs) obtained from the rectal samples cultured aerobically (A), anaerobically (B) and microaerophilically (C). Mann-Whitney U tests were used to assess statistical significance among the groups of interest, with the following significance cut-offs: p<0.05*, p<0.01**, p<0.001***. n=15 independent patients were included for these analyses.

FIG. 5. Schematic representation of methods used to culture from mucosal samples in order to obtain a eukaryotic DNA depleted metagenomic sample for metagenomic sequencing.

FIG. 6. Total Raw Read counts from 64 metagenomic samples sequenced on an Illumina HiSeq X Ten System, at 32 plex. The maximum read count generated was 33,11,896 reads, while the minimum read count generated was 20,240,944 reads, with a median of 23,928,436 reads generated.

FIG. 7. Potential human, mouse and adaptor sequence contamination rates amongst the 64 metagenomic samples. Sequence trimming of the raw reads was performed using Trimmomatic v.0.38 to ensure that all technical sequencing defects were removed and guarantee that only clean, raw reads remained. The raw reads were then mapped against the human reference genome (hg19), mouse reference genome (mm10) and adapter sequence (adapters) reference sequences using bowtie2 to eliminate the presence of any contaminating reads.

FIG. 8. Percentage of human DNA in raw versus enriched samples. Each data point represents a raw (blue) or metagenomic (red) sample obtained from the lung (circle) or nasopharynx (triangle). Data presented as mean+/−SEM. Two way ANOVA and Sidak's multiple comparison tests were performed to assess statistical significance between sample type and sample site. Statistical significance: ****p<0.0001. (n=6 raw and n=45 metagenomic samples across 7 different media types and 3 different conditions).

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, nucleic acid extraction and metagenomic sequencing).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, “depleting” means a reduction in the relative amount of host (subject) nucleic acids from the sample, or food, drink or probiotic composition. In an embodiment, during a depleting step of the invention at least 50%, at least 60%, at least 70%, at least 80%, at least 90%, at least 95%, at least 99%, and preferably all, of the host (subject) nucleic acids are removed.

As used herein, “nucleic acids” refers to any polynucleotide or a fragment thereof. The nucleic acids may be DNA or RNA, double-stranded or single-stranded, or a combination thereof. For example, nucleic acids include genomic DNA and mRNA, or amplified nucleic acids obtained directly or indirectly therefrom.

As used herein, as the name suggests, a “phenotype of interest” can be any manifestation known to be, or suspected to be, influenced by the microbiome. In one embodiment, the phenotype is a disease state, either specific such as Irritable Bowel Syndrome, or more general such as inflammation. In another embodiment, the phenotype is associated with more general traits such as health, fitness and digestion. Examples of some phenotype of interest include, but are not limited to, acne, antibiotic-associated diarrhoea, asthma, an allergy, autism, autoimmune diseases, cancer, dental cavities, depression, anxiety, diabetes, eczema, gastric ulcers, atherosclerosis, inflammatory bowel diseases, allergies, intolerance, malnutrition, obesity, hypertension, dyslipidaemia short chain fatty acid levels and dysbiosos.

### Microbiome

As used herein, “microorganisms” refers to microscopic organisms found as part of the microbiome of a region of another, larger multicellular, organism. Examples of microorganisms which may be identified using the method of the invention include bacteria (such as gram-positive bacteria, gram-positive bacterial spores, gram-negative bacteria, gram-negative bacterial spores), fungus (including fungal spores), protozoa, viruses (including bacteriophages) and viroids. Microorganisms identified by the methods of the invention are live microorganisms obtained from the subject.

As used herein, “microbiome” refers to the microorganisms of a particular region of a subject. For example, the gut microbiome refers to the community of microorganisms in the gut.

Bacteria which may be identified using the methods of the invention can be Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are gram-negative cells, and in some embodiments, the bacterial cells are gram-positive cells. Examples of bacteria which may be identified using the methods of the invention include, but are not limited to, Yersinia spp., Escherichia spp., Klebsiella spp., Bordetella spp., Neisseria spp., Aeromonas spp., Francisella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., Collinsella spp., Dorea ssp., Roseburia spp., Anaerostipes spp., Fusicatenibacter spp., Parabacteroides spp., Faecalibacterium spp., Blautia spp. or Lactobacillus spp. In some embodiments, the bacteria may be Bacteroides thetaiotaomicron, Bacteroides fragilis, Bacteroides distasonis, Bacteroides vulgatus, Bacteroides uniformis, Bacteroides cellulosilyticus, Bacteroides dorei, Bacteroides caccae, Bacteroides xylanisolvens, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinomycetemcomitans, cyanobacteria, Escherichia coli, Helicobacter pylori, Selenomonas ruminantium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, Dorea longicatena, Bifidobacterium longum, Bifidobacterium adolescentis, Collinsella aerofaciens, Roseburia faecis, Anaerostipes hadrus, Fusicatenibacter saccharivorans, Parabacteroides distasonis, Parabacteroides merdae, Blautia obeum, Faecalibacterium prausnitzii or Streptomyces ghanaenis. The methods of the invention may also identify a strain of a bacteria, such as a bacteria listed above.

Fungi which may be identified using the methods of the invention include, but are not limited to, Candida spp., such as Candida albicans, Candida tropicalis, Candida parapsilosis, Candida glabrata, Candida krusei and Candida lusitaniae, Saccharomyces spp. such as Saccharomyces cerevisiae, Penicillium spp. such as Penicillium aff. commune, Aspergillus spp. such as Aspergillus aff. versicolor, Cryptococcus spp., Malassezia spp. such as Malassezia globose, Malassezia restricta, and Malassezia pachydermatis, Cladosporium spp. such as Cladosporium aff. herbarum, Galactomyces spp. such as Galactomyces geotrichum, Debaryomyces spp. such as Debaryomyces hansenii and Trichosporon spp. The methods of the invention may also identify a strain of a fungus, such as a fungus listed above.

Protozoa which may be identified using the methods of the invention include, but are not limited to, Blastocystis spp. such as Blastocystis enterocola and Blastocystis homins, Neobalantidium coli, Entamoeba spp. such as Endolimax nana, Iodamoeba batschlii, Enterocytozoon spp. such as Enterocytozoon bieneusi, Encephalitozoon intestinalis and Encephalitozoon cuniculi, Pentatrichomonas hominis, Dientamoeba fragilis, and Giardia lamblia. The methods of the invention may also identify a strain of a protozoa, such as a protozoa listed above.

Viruses which may be identified using the methods of the invention include, but are not limited to, Adenoviridae, Anelloviridae, Astroviridae, Herpesviridae such as cytomegalovirus (CMV) and the Epstein-Barr virus (EBV), Novoviridae, Parvoviridae, Pneumoviridae, Picornaviridae and Picobirnaviridae. The methods of the invention may also identify a strain of a virus, such as a virus listed above.

As used herein, “region” refers to a portion of the organism comprising a population of microorganisms (microbiome). In animals, examples of suitable regions to be analysed include the gastrointestinal system, the respiratory system, the female reproductive system, the bladder or the skin or a portion of any one thereof. With regard to plants, an example of a suitable region to be analysed is the roots.

A sample of the region to be analysed can be obtained by any suitable means and is well within the skill of those in the art. The sample may be a liquid, solid or a combination thereof. For example, a sample of the gastrointestinal system can be obtained by biopsy from the relevant region. Such a biopsy can comprises gastrointestinal fluid, fluid associated with a mucosal surface of the gastrointestinal system, a mucosal tissue sample, or a combination of any two or more thereof. In another example, a vaginal swab can be obtained from the female reproductive system. In further example, the sample may be a faecal sample. In another example, a bronchial lavage can be obtained from the respiratory system. In yet a further example, the sample is urine. In another example, the sample is skin.

As used herein, the “subject” can be any multicellular organism which comprises at least one region which comprises a heterogeneous population of microorganisms (i.e. a microbiome). In a preferred embodiment, the subject is an animal or plant. In one example, the animal is a vertebrae. For example, the animal is a mammal, avian, arthropod, chordate, amphibian or reptile. In an embodiment, the animal is a mammal. Exemplary subjects include but are not limited to human, fish, prawns, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In an embodiment, the mammal is a human.

### Food, Drink or Probiotic Composition

As used herein, a “food” can be any substance which can be eaten by an animal, such as animals described herein. In an embodiment, the food is known to typically comprise a probiotic microorganism. In an embodiment, the food is a fermented food product. Examples of food which can be analysed using a method of the invention include, but are not limited to, yogurt, sauerkraut, kefir, kimchi, fermented vegetables, meat, natto, cheese, gerkins, brine-cured olives, tempeh, miso, cream, butter and kimchi.

As used herein, a “drink” (or beverage) can be any liquid substance which can be consumed by an animal, such as animals described herein. In an embodiment, the drink is known to typically comprise a probiotic microorganism. In an embodiment, the drink is a fermented drink product. Examples of drinks which can be analysed using a method of the invention include, but are not limited to, kombucha, coconut kefir, kvass, milk, apple cider vinegar and buttermilk.

As used herein, a “probiotic composition” is a substance, typically to be administered to an animal, which comprises probiotic microorganisms. Probiotics, in accordance with the teachings of this invention, comprise microorganisms that benefit health when consumed in an effective amount. Desirably, probiotics beneficially affect the human body's naturally-occurring gastrointestinal microflora and impart health benefits apart from nutrition. Probiotics may include, without limitation, bacteria, yeasts and fungi. In one embodiment, the probiotic composition is in the form of a capsule comprising the probiotic microorganisms. Examples of probiotics include, but are not limited to, bacteria of the genus Lactobacillus, Bifidobacteria, Streptococcus or combinations thereof, that confer beneficial effects to humans. Non-limiting examples of Lactobacillus species found in the human intestinal tract include L. acidophilus, L. casei, L. fermentum, L. saliva roes, L brevis, L. leichmannii, L. plantarum, L. cellobiosus, L. reuteri, L. rhamnosus, L. bulgaricus, and L. thermophilus. Non-limiting species of Bifidobacteria found in the human gastrointestinal tract include B. angulatum, B. animalis, B. asteroides, B. bifidum, B. bourm, B. breve, B. catenulatum, B. choerimim. B. coryneforme, B. cuniculi, B. dentiumn, B. gallicum, B. gallinarum, B indicum, B. longwn, B. magnum, B. merycicum, B. minimum, B. pseudocatemilatum, B. pseudolongwn, B. psychraerophilum, B. pullorum, B. ruminantium, B. saeculare, B. scardovil, B. simiae, B. subtile, B. thermacidophilum, B. thermophilum, and B. urinalis. Other non-limiting probiotic species include Streptococcus thermophiles, Streptococcus salivarus and Streptococcus cremoris.

### Culturing

In an embodiment, depleting the metagenomic sample of nucleic acids from the subject, or the food, drink or probiotic composition, as described herein comprises culturing in vitro microorganisms from a sample of the microbiome from the region of the subject, or of the food, drink or probiotic composition. The culturing conditions may not be suitable for every microorganism present in the sample due to factors such as oxygen level and nutrients in the culture media. Nonetheless, the skilled person will appreciate the type of microorganisms that a specific type of culturing step supports.

In an embodiment, a sample is taken from the food, drink or probiotic composition of analysis using a method of the invention.

In an embodiment, the sample is cultured under aerobic conditions. As used herein, “aerobic conditions” refers to culturing the microorganisms in the presence of oxygen at or above the partial pressure of atmospheric oxygen (O₂). In an example, the microorganisms are cultured in the presence of oxygen (O₂) at or above 21%.

In an embodiment, the sample is cultured under anaerobic conditions. As used herein, “anaerobic conditions” refers to culturing the microorganisms in presence of very small amounts of oxygen (such as less than 1%), preferably in the absence of oxygen. In an embodiment, the “anaerobic conditions” comprises between about 5% and about 15%, or between about 7.5% and about 12.5%, or about 10% CO₂, between about 5% and about 15%, or between about 7.5% and about 12.5%, or about 10% H₂ and between about 5% and about 15%, or between about 7.5% and about 12.5% or about 10% CO₂, and between about 70% and about 90%, or between about 75% and about 85%, or about 80% N₂.

In an embodiment, the sample is cultured under microaerophilic conditions. As used herein, “microaerophilic conditions” refers to culturing the microorganisms of the microbiome of a region of a subject in the presence of oxygen at a lower partial pressure than that of atmospheric oxygen (O₂). In an example, the microorganisms are cultured in the presence of oxygen (O₂) below 21%. In an example, the microorganisms are cultured in the presence of 5% to 10% oxygen (O₂). In an example, the microorganisms are cultured in the presence of 2% to 10% oxygen (O₂).

A wide variety of different media can be used for the culturing depending on the types of microorganisms that are preferred to grown. The media may be liquid or solid. The selection of a suitable media for a given microbiome sample, and the type of microorganism to be identified, is well within the skill of those in the art. However, it is preferred that the media is a broad-spectrum non-selective culturing media which can be used to culture a wide variety different species and strains of microorganism. Examples of broad-spectrum non-selective culturing media include, but are not limited to, yeast-extract-casitone-fatty acid (YCFA) agar, Fastidious Anaerobic Agar (Thermo Scientific™ PB0225A), Cooked Meat Medium (Thermo Scientific™ CM0081), Wilkins Chalgren anaerobe agar (Thermo Scientific™ PB0113 or Amyl Media AM217), Brain Heart Infusion Media (BHI) (Thermo Scientific™ CM1135 or Amyl Media AM12), Antibiotic Agar No 1 for microbiology (Sigma product #70181, Thermo-Fisher Scientific PP2039), Bryant and Burkey Medium for microbiology (Sigma product #91903), CASO Agar for microbiology (Sigma product #22095), DEV Nutrient Agar for microbiology (Sigma product #41338), LB Broth Vegitone for microbiology (Sigma product #28713), Milk Agar for microbiology (Sigma product #70147), Nutrient Agar No 2 Vegitone for microbiology (Sigma product #04163), Plate Count Agar Vegitone for microbiology (Sigma product #19718), Vegitone Casein Soya Broth for microbiology (Sigma product #41298), Vegitone infusion broth for microbiology (Sigma product #41960), fastidious anaerobe agar with 5% defibrinated horse blood (Thermo Scientific™ PB0252A), and chocolate agar (Thermo Scientific™ PP2002).

In an embodiment, the broad-spectrum non-selective culturing media is yeast-extract-casitone-fatty acid (YCFA), Brain Heart Infusion Media (BHI), Anaerobic agar (ANAE), Chocolate agar, Fastidious anaerobe agar (FAA), Fastidious anaerobe agar with 5% defibrinated horse blood (FAHB) or Wilkin's-Chalgren anaerobe agar (WILK).

In an embodiment, the microorganisms are cultured on yeast-extract-casitone-fatty acid (YCFA) agar. “Yeast-extract-casitone-fatty acid (YCFA) agar” is an enriched nonselective media used in the isolation and cultivation of a wide variety of bacteria found in the human gut. The basic nutritive components of this media come from yeast extract and pancreatic digest of casein. This basal medium is then enriched with various specific vitamins, sugars, and fatty acids to ensure growth of even the most fastidious gut microbes. This media is prepared, dispensed, and packaged under oxygen-free conditions to prevent the formation of oxidized products prior to use. In one example, YCFA comprises acid casein peptone (10 g/l), ammonium sulfate (0.9 g/l), yeast extract (2.5 g/l), sodium chloride (0.9 g/l), sodium bicarbonate (4.0 g/l), magnesium sulfate (0.09 g/l), calcium chloride (0.09 g/l), D(+) glucose (2.0 g/l), dipotassium phosphate (0.45 g/l), monopotassium fosfate (0.45 g/l), starch (2.0 g/l), L-Cysteine HCl (1.0 g/l), resazurin (0.001 g/l), hemin (0.01 g/l), cellobiose (2.0 g/l), acetic acid (2.026 ml), propionic acid (0.715 ml), n-valeric acid (0.119 ml), iso-valeric acid (0.119 ml), iso-butiric acid (0.119 ml), agar (10 g/l). Another example of an YCFA preparation is provided in Example 1.

In an example, the Brain Heart Infusion Media comprises brain infusion solids (12.5 g/l), beef heart infusion solids (5 g/l), proteose peptone 10 g/l), sodium chloride (5 g/l), glucose (2 g/l), di-sodium phosphate (2.5 g/l) and agar (10 g/l).

In an example, the anaerobic agar comprises pancreatic digest of casein (20 g/l), sodium chloride (5 g/l), dextrose (10 g/l) agar (20 g/l), sodium thioglycollate (2 g/l), sodium formaldehyde sulfoxylate (1 g/l) and methylene blue (2 mg/l).

In an example, the Chocolate agar comprises proteose peptone (15 g/l), sodium chloride (5 g/l), dipotassium phosphate (4 g/l), monopotassium phosphate (1 g/l), corn starch (1 g/l), bovine haemoglobin (10 g/l), agar (10 g/l) and KoEnzyme enrichment (10.0 ml/). In an embodiment, the KoEnzyme enrichment comprises dextrose (10 g/l), L-Cysteine, HCl (2.59 g/l), L-Glutamine (1.01 g/l), L-Cystine (0.11 g/l), adenine (0.101 g/l), nicotinic adenine dinucleotide (25 mg/l), cocarboxylase (10 mg/l), guanine hydrochloride (3 mg/l), ferric nitrate (2 mg/l), p-Aminobenzoic Acid (1.3 mg/l), Vitamin B12 (1 mg/l) and thiamine (0.3 mg/l).

In an example, the Fastidious anaerobic agar comprises peptone mix (23 g/l), sodium chloride (5 g/l), soluble starch (1 g/l), agar No. 2 (12 g/l), sodium bicarbonate (0.4 g/l), glucose (1 g/l), sodium pyruvate (1 g/l), cysteine HCl monohydrate (0.5 g/l), haemin (0.01 g/l) vitamin K (0.001 g/l), L-Arginine (1 g/l), soluble pyrophosphate (0.25 g/l) and sodium succinate (0.5 g/l).

In an example, the Fastidious anaerobe agar with 5% defibrinated horse blood (FAHB) comprises peptone mix (23 g/l), sodium chloride (5 g/l), soluble starch (1 g/l), agar No. 2 (12 g/l), sodium bicarbonate (0.4 g/l), glucose (1 g/l), sodium pyruvate (1 g/l), cysteine HCl monohydrate (0.5 g/l), haemin (0.01 g/l) vitamin K (0.001 g/l), L-Arginine (1 g/l), soluble pyrophosphate (0.25 g/l), sodium succinate (0.5 g/l) and defibrinated horse blood (50 ml/l).

In an example, the Wilkin's-Chalgren anaerobe agar (WILK) comprises tryptone (10 g/l), gelatin peptone (10 g/l), yeast extract (5 g/l), glucose (1 g/l), sodium chloride (5 g/l), L-arginine (1 g/l), sodium pyruvate (1 g/l), menadione (0.0005 g/l), haemin (0.005 g/l) and agar 10 g/l).

The microorganisms can be cultured at any suitable temperature. Typically, the temperature will be about the same as the region (source) from which they were obtained. For example, for samples obtained from inside a warm blooded animal, such as the gastrointestinal system, it is generally preferred that the microorganisms are cultured at about 37° C. In another example, for samples obtained from an external surface, such as skin, of a subject, it is generally preferred that the microorganisms are cultured at about room temperature such as about 20° C. to about 25° C. In an embodiment, a sample taken from a food, drink or probiotic composition is cultured at about room temperature such as about 20° C. to about 25° C.

The microorganisms can be cultured for any suitable length of time. Ideally, the length of time is selected to ensure as much as possible of the subject's (hosts) nucleic acids are removed as reasonably possible. In one example, the microorganisms are cultured for about 3 hours to about 72 hours, or for about 12 hours to about 48 hours, or for about 18 hours to about 36 hours. In one example, the microorganisms are cultured for at least 3 hours. In one example, the microorganisms are cultured for at least 4 hours. In one example, the microorganisms are cultured for at least 5 hours. In one example, the microorganisms are cultured for at least 6 hours. In one example, the microorganisms are cultured for at least 7 hours. In one example, the microorganisms are cultured for at least 8 hours. In one example, the microorganisms are cultured for at least 10 hours. In one example, the microorganisms are cultured for at least 15 hours. In one example, the microorganisms are cultured for at least 20 hours. In one example, the microorganisms are cultured for at least 24 hours. In one example, the microorganisms are cultured for at least 30 hours. In one example, the microorganisms are cultured for at least 36 hours. In one example, the microorganisms are cultured for at least 42 hours. In one example, the microorganisms are cultured for at least 48 hours. In one example, the microorganisms are cultured for at least 60 hours. In one example, the microorganisms are cultured for at least 72 hours.

In an embodiment, the microorganisms are cultured to between about 1×10⁴ CFUs/g to about 1×10⁸ CFUs/g or about 1×10⁵ CFUs/g to about 1×10⁷ CFUs/g.

### Depletion/Selection Using Probes

In an embodiment, depleting the metagenomic sample from the subject as described herein comprises

i) hybridizing a probe to DNA of the subject in the metagenomic sample, and depleting the sample of DNA bound to the probe, and/or

ii) hybridizing a probe to DNA of microorganisms expected to be present in the metagenomic sample, and selecting DNA bound to the probe.

In a further embodiment, the method comprises culturing in vitro microorganisms from a sample of the microbiome from the region of the subject, followed by one or both of

i) hybridizing a probe to DNA of the subject in the metagenomic sample, and depleting the sample of DNA bound to the probe, and/or

ii) hybridizing a probe to DNA of microorganisms expected to be present in the metagenomic sample, and selecting DNA bound to the probe.

The use of probes to select or deplete a sample of target nucleic acids is known in the art. One or more probes may be used in the methods of the invention which hybridize to different nucleic acids.

As used herein, “probe” refers to a molecule which can be used to hybridize selectively to a target nucleic acid. Such a probe useful for the present invention has also been referred to in the art as a bait or capture probe.

The probe may comprise or may consist of RNA, DNA, PNA (peptide nucleic acid), LNA (locked nucleic acid) and/or other analogs. In particular analogs of the nucleobase T or U may be used insofar as they allow for hybridization with A residues. In an embodiment, the probe is a synthetic single stranded oligonucleotide of any suitable length such as least 10, at least 12, at least 14, at least 16, at least 18, at least 20, at least 25, at least 30, at least 35, at least 40 nucleotides long. According to one embodiment, at least 60%, at least 70%, at least 80%, at least 90%, at least 95% or at least 100% of all pairing units (e.g. nucleobases) of the probe are capable of hybridizing to a target nucleic acid.

A set of probes may be used for targeting different nucleic acids. In one embodiment, the probes may be prepared from the whole genome of the target organism (subject), for example, where the probes are prepared by a method that includes fragmenting genomic DNA of the target organism (e.g., where the fragmented sequences are end-labeled with oligonucleotide sequences suitable for PCR amplification or DNA sequencing or where the sequences are prepared by a method including attaching an RNA promoter sequence to the genomic DNA fragments and preparing the probe by transcribing (e.g., using biotinylated ribonucleotides) the DNA fragments into RNA). Alternatively, the probe(s) may be prepared from specific regions of the target organism genome (e.g., are prepared synthetically).

The probe may be present in a hybridization composition in free form. The probe may then be immobilized to a solid phase during or after the hybridization reaction. The probe may also be labeled with a compound that reacts with a second compound that in turn is immobilized to a solid support.

Alternatively, the probe is provided in an immobilized form wherein the probe is attached to a solid support. Preferably, a solid support functionalized with the probe is used and hence is comprised in a hybridization composition. Immobilization to the solid support may be achieved using techniques that are well-known and standard in the art. In some embodiments, the probe is attached to a solid support using a linker structure.

The solid support may be provided by various materials, including but not limited to reaction vessels, microtiter plates, particles, magnetic particles, cellulose, columns, plates, membranes, filter papers and dipsticks or any other solid support that can be used in separation technologies. Any support can be used as long as it allows separation of a liquid phase. Different solid supports were also used in known methods for selecting/depleting nucleic acids.

In one embodiment, the solid support is provided by particles commonly also referred to as beads. The particles used may be made of glass, silica, polymers, polystyrene-latex polymers, cellulose and/or plastic. According to a preferred embodiment, the solid support is provided by a suspension of particles that are functionalized with the probe. The use of magnetic particles is preferred. When using magnetic particles as solid support, they may have superparamagnetic, paramagnetic, ferrimagnetic or ferromagnetic properties. Respective magnetic particles can be easily separated by the aid of a magnetic field, e.g. by using a permanent magnet and therefore have advantages with respect to the processing. They are compatible with established robotic systems capable of processing magnetic particles. Here, different robotic systems exist that can be used to process the magnetic particles to which the hybrids of the probe and the nucleic acid are bound. According to one embodiment, magnetic particles are collected at the bottom or the side of a reaction vessel and the remaining liquid sample is removed from the reaction vessel, leaving behind the collected magnetic particles to which the hybrids are bound. Removal of the remaining sample can occur by decantation or aspiration. In an alternative system the magnet, which is usually covered by a cover or envelope, plunges into the reaction vessel to collect the magnetic particles. In a further alternative system, the sample comprising the magnetic particles can be aspirated into a pipette tip and the magnetic particles can be collected in the pipette tip by applying a magnet e.g. to the side of the pipette tip. The remaining sample can then be released from the pipette tip while the collected magnet particles which carry the bound hybrids remain due to the magnet in the pipette tip. The collected magnetic particles can then be processed further. Such systems are also well-known in the prior art and are also commercially available (e.g. BioRobot EZ1, QIAGEN). Also other processing systems are known and can be used.

Particles may also be separated by filtration, centrifugation or by using spin columns that can be e.g. loaded with a suspension of particles as is well-known to the skilled person. When the solid support is centrifuged it may be pelleted or passed through a centrfugible filter apparatus or column.

In some embodiments, the probe may be biotinylated or otherwise labeled so as to facilitate separation of the hybrids. Biotin can be derivatized to probe nucleotides, for example using linkers, without impairing the ability of the probe to hybridize to the target nucleic acid. Because biotin reacts with avidin/streptavidin, avidin or streptavidin may be employed in conjunction with a biotinylated probe. The avidin or streptavidin may be linked to a solid support, such as particles or the surface of a vessel where it may bind the biotinylated probe. The solid support may then be separated from the remainder of the sample e.g. by removing the solid support from the remaining sample or vice versa to isolate the biotinylated probe, which itself is hybridized to the target nucleic acid. The probe can also be labelled for separation using a number of different modifications that are well known to those of skill in the art. Non-limiting alternatives include labelling the probe with an epitope tag and utilizing an antibody or a binding fragment thereof that recognizes that epitope for capture, for example, labelling the probe with digoxigenin and using an anti-digoxigenin antibody for capture. Furthermore, haptens may be used for conjugation e.g. with nucleotides or oligonucleotides. Commonly used haptens for subsequent capture include biotin (biotin-11-dUTP), dinitrophenyl (dinitrophenyl-11-dUTP). These modifications include for example fluorescent modifications. Commercially available fluorescent nucleotide analogs that may be incorporated include but are not limited to Cy3™-dCTP, Cy3™-dUTP, Cy™ 5-dCTP, fluorescein-12-dUTP, AlexaFluor® 594-5-dUTP, AlexaFluorR™.-546-14-dUTP and the like. Fluorescein labels may also be used as a separation moiety using commercially available anti-fluorescein antibodies. Also suitable is the labelling with radioisotopes, enzyme labels and chemiluminescent labels.

Furthermore, in case the probe itself is not linked to a solid support, hybrid binding agents immobilized to a solid support may be used to facilitate separation of the formed hybrids, such as e.g. anti-hybrid binding agents such as anti-DNA/RNA antibodies or binding fragments thereof. Such embodiments are e.g. suitable in case a RNA/DNA hybrid is formed upon hybridization of the capture probe to the nucleic acid. A respective hybrid binding agent could likewise be immobilized to a solid support according to the principles described above.

Thus, many established systems are available that achieve that hybrids formed between the probe and the nucleic acid are eventually immobilized onto a solid support which facilitates the separation of the hybrids.

### Metagenomics

Metagenomics entails a study of multiple genomes from different organisms, and can be applied to profile the genomes of a community of microorganisms. For example, a metagenomic analysis can be used to determine the sequence and to measure the abundance of genomes of multiple microorganisms within a single sample (see, for example, Metagenomic Analysis and its Applications in Encyclopedia of Bioinformatics and Computational Biology, Ed by Ranganathan et al. Elsevier, 2019).

As used herein, the term “metagenomic sample” refers to a composition comprising genomic nucleic acids obtained from at least a sub-population of the microorganisms of the microbiome of the region. The microorganisms may or may not have been cultured. In an embodiment, the nucleic acids are DNA or RNA or a combination thereof. In a preferred embodiment, the nucleic acids comprise or consist of genomic DNA.

As used herein, the term “obtaining a metagenomic sample” refers to any means of coming into possession of the sample. The sample may have been prepared by another party, such as purchased therefrom, a collaborator or a business partner. In an embodiment, the step includes processing the microorganisms and extracting nucleic acids therefrom using standard procedures.

As used herein, “metagenomic sequencing” refers to a process wherein which nucleic acids of a metagenomic sample is subjected to nucleic acid sequencing (see, for example, Sharpton, 2014; Wang et al., 2015; Quince et al., 2017; Kumar et al., 2017). Metagenomic sequencing can be achieved using any method known in the art such as by next-generation sequencing (NGS). In an embodiment, the Illumina HiSeq X Ten System is used for metagenomics sequencing.

As the skilled person will appreciate, the portion analysed will need to be of sufficient length to classify the source of the nucleic acid gene.

In an embodiment, metagenomic analysis following metagenomic sequencing typically comprises the assembly, identification and/or quantification of genomes of microorganisms in a sample. In an example, a taxonomically organised k-mer based sequence database will be generated from genome sequences of single bacteria. Metagenomic reads are then assigned based on sequence identity to determine sample species composition (e.g. Kraken described by Wood and Salzberg (2014) and https://genomebiology.biomedcentral.com/articles/10.1186/gb-2014-15-3-r46 and Forster et al. (2019)). In another example, metagenomic reads may be directly assembled to generate metagenome assembled genomes (MAGs) with species composition and proportion determined through prevalence of these sequences within the complete sample (see, for example, Almeida (2019)).

As used herein, “reference genome” refers to a genetic sequence for a particular organism with which other sequenced genomes can be compared. In an example, sequence comparison can be performed using BLAST, Megablast, BLAT and SSAHA.

In an example, metagenomic analysis can be performed using Integrated Microbial Genomes and Metagenomes System (IMG/M) (http://img.jgi.doe.gov/m) or Metagenomic Rapid Annotations using Subsystems Technology (MG-RAST).

In an embodiment, the metagenomic sequencing comprises the sequencing of at least one pre-selected region of a nucleic acid such as genomic DNA. Preferably, there is a database of sequences for the pre-selected covering a wide range of species of microorganisms, and strains thereof, to enhance the chances a microorganisms from the microbiome being identified. Sequences obtained can be compared to known databases such as SILVA (https://www.arb-silva.de/) and GenBank (https://www.ncbi.nlm.nih.gov/genbank/). In an embodiment, the full length of a gene is sequenced.

Identifying a Microorganism Associated with a Phenotype of Interest

The methods of the invention can be used to identify a microorganism which may be associated with a phenotype of interest. Typically, such methods are performed using a suitable number of case (with the phenotype) and control (without the phenotype) samples from different subjects. Case/control studies for identifying factors which influence a phenotype are well known in the art. In one example, the methods of the invention can be used in microbiome genome association studies which are described in Awany et al. (2019).

In another example, the comparison of samples derived from phenotypically different sources can be compared directly to identify differences. Case samples sourced from sites of inflammation can be compared to control samples derived from sites without inflammation.

In an embodiment, the method includes performing a selection step to assist in identifying microorganisms that possess the phenotype of interest. Such selection methods are well known in the art. In one example, antibiotic resistance might be selected by inclusion of the antibiotic within the culture media. In a second example, exposure of samples to ethanol selection prior to culturing will select for spore forming bacteria (Browne, 2016).

### EXAMPLES

### Example 1—Methods

### Sample Collection

Gastrointestinal biopsies were obtained during paediatric endoscopy lists at Monash Children's Hospital, Melbourne, Australia, from patients receiving clinically indicated colonoscopies. Samples were obtained from three bowel regions (Terminal Ileum, Caecum and Rectum), with two biopsies acquired from each site. The mucosal samples were transported on wet ice from the theatres to the laboratory at 4° C.

### Bacterial Culturing

To prepare the samples for bacterial culturing, they were weighed, diluted by a factor of 10 with pre-reduced (anaerobic) PBS, serially diluted to 10-6 and plated directly onto yeast-extract-casitone-fatty acid (YCFA) agar. Bacteria were cultured under aerobic, anaerobic and microaerophilic conditions.

YCFA agar was prepared as follows:

### YCFA Agar

| Ingredient | Amount required | |
|---|---|---|
| Before Autoclaving | | |
| Tryptone | 10 | g/L |
| NaHCO3 (Sodium Bicarbonate) | 4 | g/L |
| Yeast Extract | 2.5 | g/L |
| (D) + Glucose | 2 | g/L |
| (D) + Maltose | 2 | g/L |
| (D) + Cellobiose | 2 | g/L |
| L-cysteine | 1 | g/L |
| YCFA Mineral Solution 1 | 150 | ml/L |
| YCFA Mineral Solution 2 | 150 | ml/L |
| Vitamin Solution 1 | 1 | ml/L |
| Haemin Solution | 10 | ml/L |
| Resazurin Solution | 1 | ml/L |
| VFA Mix | 6.2 | ml/L |
| Bacterial Agar | 8 | g/L |
| dd•H2O | make up to 1 L | |
| Comments | Adjust pH to 7.45 and autoclave | |
| After autoclaving | | |
| Vitamin Solution 2 | 1 | ml/L |

### Mineral Solution 1

| Ingredient | Amount required |
|---|---|
| K₂HPO₄ Potassium Phosphate Dibasic | 3 g/L |
| dd•H₂O | Make up to 1 L |

### Mineral Solution 2

| Ingredient | Amount required | |
|---|---|---|
| KH₂PO₄ Potassium Phosphate | 3 | g/L |
| (NH₄)₂SO₄ Ammonium Sulphate | 6 | g/L |
| NaCl Sodium Chloride | 6 | g/L |
| MgSO₄ Magnesium Sulphate | 0.6 | g/L |
| CaCl₂ (dry) Calcium Chloride | 0.6 | g/L |

| | |
|---|---|
| dd•H₂O | Make up to 1 L |

### Vitamin Solution 1

| Ingredient | Amount required | |
|---|---|---|
| Biotin | 5 | mg/L |
| Vitamin B12 | 5 | mg/L |
| 4-Aminobenzoic Acid | 15 | mg/L |
| Folic Acid | 25 | mg/L |
| Pyridoxine | 75 | mg/L |
| dd.H₂O | Make up to 1 | L |

### Vitamin Solution 2

| Ingredient | Amount required | |
|---|---|---|
| Thiamine | 50 | mg/L |
| Riboflavin | 50 | mg/L |
| dd.H₂O | Make up to 1 | L |

### Haemin Solution

| Ingredient | Amount required | |
|---|---|---|
| KOH Potassium Hydroxide Powder | 2800 | mg/L |
| Ethanol (>95%) | 250 | mg/L |
| Haemin | 1000 | mg/L |
| dd.H2O | make up to 1 | L |

### Resazurin Solution

| Ingredient | Amount required | |
|---|---|---|
| Resazurin | 1000 | mg/L |
| dd.H2O | make up to 1 | L |

### Volatile Fatty Acid (VFA) Solution

| Ingredient | Amount required | |
|---|---|---|
| Acetic acid | 653.8462 | ml |
| Propionic acid | 230.7692 | ml/L |
| n-Valeric acid | 38.4615 | ml/L |
| Isovaleric acid | 38.4615 | ml/L |
| Isobutyric acid | 38.4615 | ml/L |

### Anaerobic Culturing

Plates for culturing of anaerobic bacteria were incubated at 37° C. in the Whitely A95 anaerobic workstation (Don Whitley Scientific; Yorkshire, United Kingdom), containing 10% carbon dioxide, 10% hydrogen and 80% nitrogen.

### Aerobic Culturing

Plates for culturing of aerobic bacteria were incubated at 37° C., in ambient oxygen.

### Microaerophilic Culturing

Plates for culturing of microaerophilic bacteria were stored in 2.5 L gas jars (Thermo Scientific; Waltham, Mass., United States) containing 2.5 L CampyGen gas packs (Oxoid; Basingstoke, Hampshire, United Kingdom), and incubated at 37° C.

### Colony Counting

To enable enumeration of colony forming unit (CFU) counts between samples, 10 μl aliquots of each dilution factor were plated in triplicate onto YCFA agar. Plates were enumerated following a 24-hour incubation in appropriate environments. Calculations were performed to determine CFU counts per gram of mucosal tissue.

### Culturing for Metagenomic Analysis

To culture for metagenomic analysis, 50 μl aliquots of each dilution factor, prepared from the whole mucosal sample, were applied to YCFA agar plates and uniformly spread across the plate using disposable plate spreaders (International Scientific Group). Plates were incubated at 37° C., in appropriate environments. Plates were scraped for metagenomic analysis 24-hours after plating, using plates harbouring distinct, non-converging bacterial colonies.

To prepare samples for metagenomic analysis, 600 μl of pre-reduced PBS was applied to the surface of the plate, and sterile spreading loops (International Scientific Group) were used to disrupt bacterial colonies from the YCFA agar and suspend them in PBS. The bacterial suspension was transferred into a 1.7 ml Eppendorf tube and stored at −80° C., prior to DNA extraction.

### DNA Extraction

Genomic DNA was extracted from bacterial samples using the MP Biomedicals FastDNA® SPIN Kit for soil (MP Biomedicals; Santa Ana, Calif., USA), optimised for DNA extraction from bacterial samples.

Bacterial samples were thawed. Lysing Matrix E (LME) tubes (MP Biomedicals) were filled with 978 μl of Sodium Phosphate Buffer solution (MP Biomedical), 122 μl of MT Buffer (MP Biomedical) and 300 μl of the corresponding bacterial culture. Samples were homogenized at 1600 rpm for 40 seconds in the FastPrep96® high-throughput homogenizer (MP Biomedicals). They were then centrifuged for 10 minutes at 21000×g, room temperature, and the supernatant was transferred to 1.7 ml Eppendorf tubes, containing 250 μl of protein precipitation solution (PPS, MP Biomedicals). The tubes were inverted by hand 10 times, and centrifuged for five minutes at 21000×g, room temperature.

The supernatant was transferred into 15 ml falcon tubes, containing 1 ml of Binding Matrix solution (MP Biomedical). Tubes were inverted by hand for 2 minutes and allowed to settle for 5 minutes. 850 μl of supernatant was discarded from each tube without disrupting the settled binding matrix, and the binding matrix was resuspended in the remaining supernatant. Following resuspension, 700 μl of supernatant was transferred into corresponding Spin Filters and collection tubes (MP Biomedicals), which were centrifuged for 1 minute at 21000×g, room temperature, and flow through was discarded. This process was repeated until all of the solution had been passed through the Spin Filter. 500 μl of SEWS-M wash buffer (MP Biomedicals) was applied to the Spin Filters, the binding matrix was resuspended via pipette action, and centrifuged for 1 minute at 21000×g, room temperature. The flow through was discarded. The samples were centrifuged for 2 minutes at 21000×g, room temperature, and placed into new catch tubes.

To elute the DNA, 50 μl of DEPC water (Thermo-Fisher Scientific) was applied to each Spin Filter. The samples were allowed to stand for 5 minutes, then centrifuged for 1 minute at 21000×g, room temperature. The DNA (ng/μl) in each sample was quantified using the NanoDrop ND-100 Spectrophotometer (Analytical Technologies; Wilmington, Del., USA), and DNA products were stored at −20° C.

### Metagenomic Sequencing

Metagenomic samples were sequenced using the Illumina HiSeq X Ten System, at 32 plex. Samples were prepared to ensure that they contained at least 1.5 μg of intact genomic DNA at a concentration of at least 20 ng/μl, made up to a volume of at least 15 μl using nuclease free water.

Following the completion of sequencing, preliminary analyses were performed to determine the sequencing quality provided. The number of clean reads and bases obtained, read lengths, GC content (%), quality scores and sequence complexity distributions were analysed to give an initial indication of the sequencing quality. Initial Quality Control (QC) procedures are important in order to identify potential contamination and filter out sequencing artefacts, including low-quality or contaminating raw read. Low-quality sequences, along with sequencing artefacts and contamination, significantly affect the deductions that can be derived from the data and commonly result in erroneous conclusions. Therefore, these initial steps are of fundamental importance.

Following assurance that the sequencing data was of sufficient quality to continue analyses, sequence trimming was performed with Trimmomatic v.0.38. This step was essential to ensure that all technical or adaptor sequences were removed and guarantee that low-quality or contaminating reads were filtered out so that only the clean raw reads remained. Following trimming with Trimmomatic, FastQ Screen (version 0.13.0) was used to map our library of sequences against human, mouse and adapter sequence databases. Mapping of our sequencing data to the Human reference genome (hg19), mouse reference genome (mm10) and adapter sequences (adapters) using bowtie2, enabled identification of any remaining contaminating reads. This process is particularly important to consider with regards to potential human contamination, as this project represents the unique culturing for metagenomic purposes has been performed from human biopsy samples.

### Example 2—Comparison of Sample States

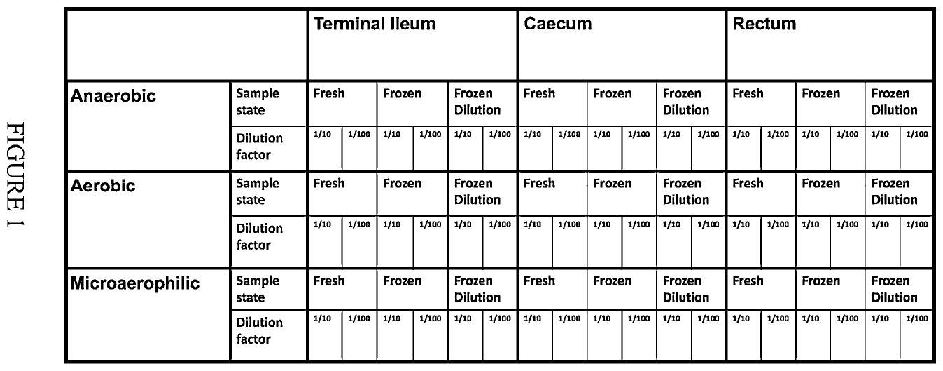

To determine differences in bacterial viability between fresh and frozen (diluted and whole) samples, several sample formulations were prepared (FIG. 1), and CFUs were compared. Two initial dilution factors were performed ( 1/10 and 1/100) to assess the effect of a greater initial dilution on bacterial dissociation from biopsies and bacterial yields obtained.

Consistently high inter-sample variability in the density of bacterial colonisation was found across the three bowel regions and culturing environments, with co-efficients of variation ranging from 99.88% to 410.32%. Freezing generally decreased bacterial viability, with freezing diluted samples causing greater bacterial loss than freezing whole biopsies. Significant losses were noted among anaerobic samples, regardless of the bowel region, while general, but not always significant, trends were noted for the aerobic and microaerophilic samples. Additionally, anaerobic culturing (6.12×10⁶ CFUs/g) consistently achieved greater bacterial counts among all sample types, compared to aerobic (1.35×10⁵ CFUs/g) and microaerophilic (1.43×10⁵ CFUs/g) culturing, likely resulting from the dominance of anaerobic bacteria in the gastrointestinal tract. CFUs from various sample preparations were compared (FIG. 1), and differences among these comparators were noted between sample types and culturing environments.

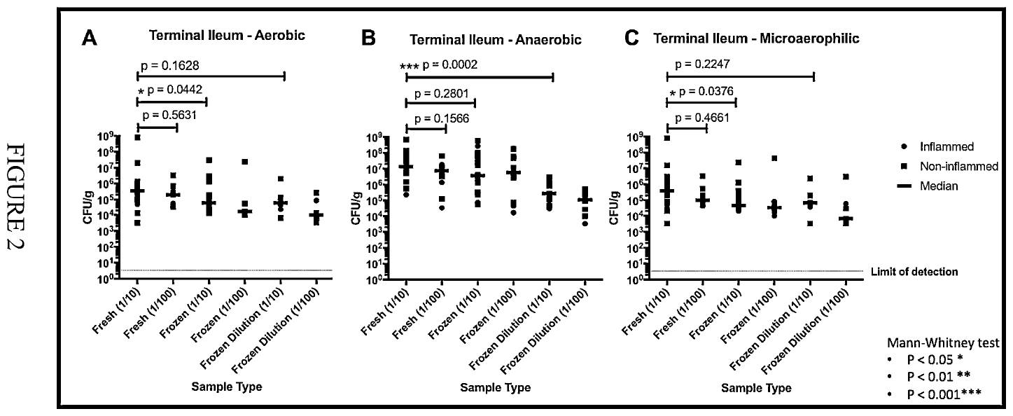

For terminal-ileal samples (FIG. 2), comparison of fresh and frozen whole samples found freezing to significantly impact bacterial viability recovered, in aerobic (p=0.0442) and microaerophilic (p=0.0376) environments. Similar trends were noted anaerobically, with freezing diluted samples having a more significant impact on bacterial viability than freezing whole samples (p=0.0002). Therefore, freezing whole and diluted terminal ileal samples significantly affects bacterial viability, in a culturing-environment dependent manner, with sample dilution causing greater bacterial loss.

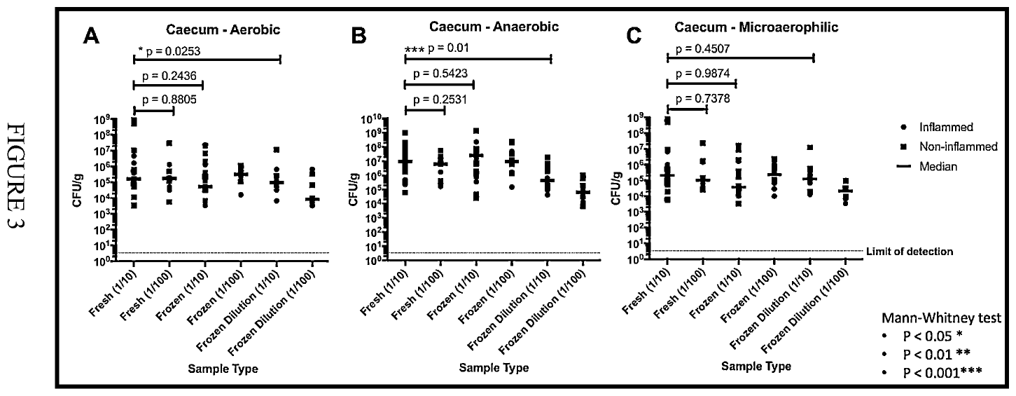

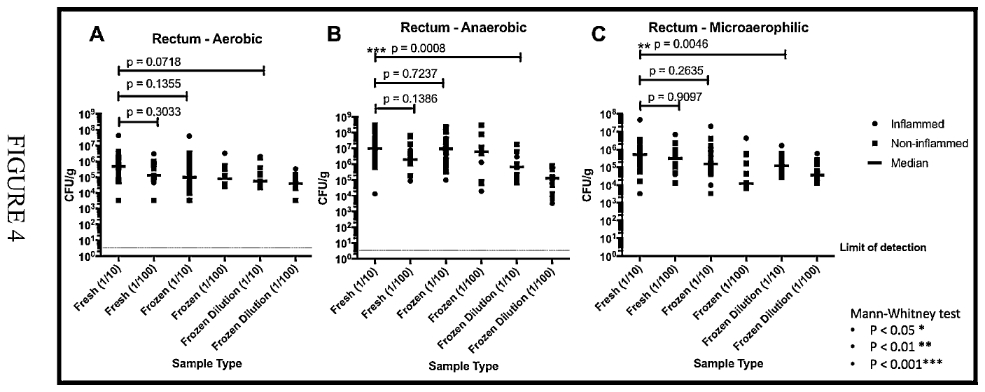

Freezing whole mucosal caecal (FIG. 3) and rectal (FIG. 4) samples had no significant impact on bacterial viability recovered, in any culturing-environment. However, compared to fresh samples, freezing diluted samples had a significant impact on bacterial recovery when cultured anaerobically (caecum p=0.01, rectum p=0.0008). Similar trends were noted among caecal samples cultured aerobically (p=0.0253) and rectal samples cultured microaerophilically (p=0.0046). Additionally, bacterial yields were generally dependent on the initial dilution, with a larger ( 1/100) initial dilution impairing the bacterial density and diversity recovered.

Overall, freezing of biopsies affects bacterial yields in a tissue-type and culturing-environment dependent manner, with original bacterial density and diversity likely influencing the outcome. The tissue-type specific effects seen are probably the result of intrinsic differences in tissue and microbial composition across the regions of the bowel.

### Example 3—Diverse Bacterial Communities Cultured

From the first 70 patients recruited to this study, 2487 isolates have been cultured, from which 2292 high quality sequences were generated via 16S rRNA sequencing. These partial-length 16S rRNA sequences were aligned against the NCBI blast database, and taxonomic classification of isolates was based on gene homology to previously sequenced organisms, to define a characterized or candidate novel isolate. Conservative cut-offs (Table 1) were used to classify novelty, as these results were based on partial-length 16S rRNA sequences, rather than full-length 16S rRNA sequences or whole-genome sequences. This work has allowed for preliminary identification of 1095 known and 1381 putative novel isolates.

TABLE 1
Sequence similarity cut-offs for defining novelty

| | Sequence | Sequence similarity |
|---|---|---|
| | similarity | cut-offs for |
| | cut-offs | full-length |
| | used here | 16 S sequences |
| Species level | <96% | <98.7% |
| Genus level | <90% | <93% |
| Family level | <80% | <80% |

A phylogenetic tree was constructed to visualise relationships between species (not shown). Of the isolates accurately classified, the Bacteroidetes phylum dominated (n=1243), followed by the Firmicutes (n=687), Proteobacteria (n=435), and Actinobacteria (n=122) phyla. Data was overlayed onto the phylogeny to visualise the distribution of isolates obtained from inflamed (n=851) and non-inflamed (n=1441) biopsies.

### Example 4—Metagenomic Sequencing of Cultured Bacterial Communities from Mucosal Biopsy Samples

Previously, metagenomic sequencing of microbial communities from mucosal samples proved hugely challenging as the inability to isolate bacteria from the human mucosa resulted in a very costly and inefficient sequencing process. This was largely due to the great number of contaminating human DNA reads amongst bacterial reads.

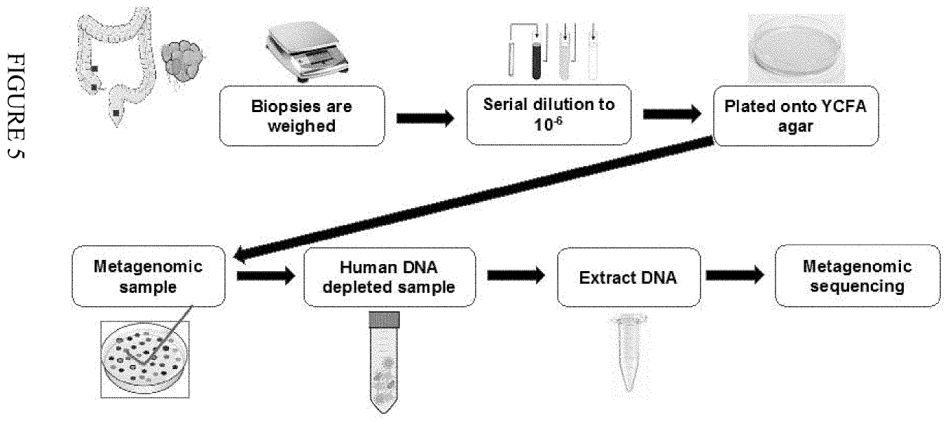

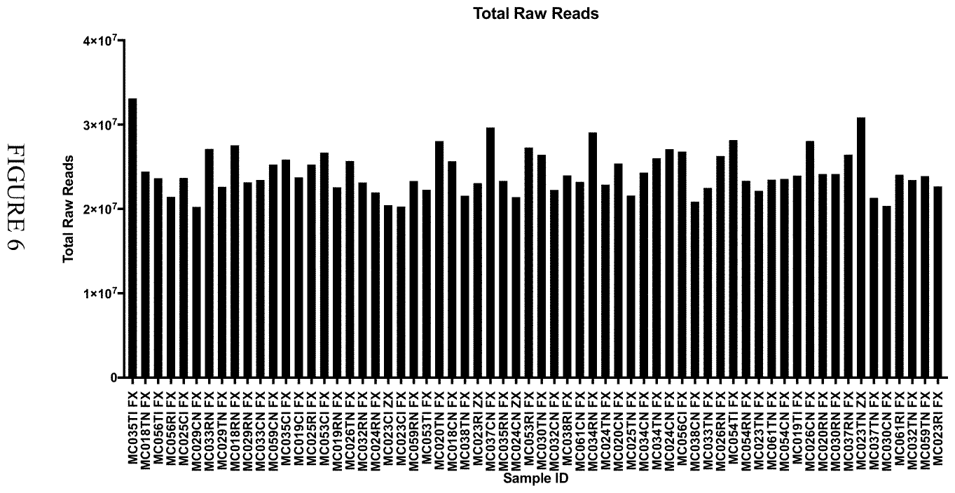

Metagenomic sequencing was performed on 64 samples generated via culturing from human biopsies (FIG. 5). These samples were sequenced on an Illumina HiSeq X Ten System, at 32 plex. The maximum read count generated was 33,11,896 reads, while the minimum read count generated was 20,240,944 reads, with a median of 23,928,436 reads generated (FIG. 6). This represents on average greater than 50× coverage of the bacterial genomes within the metagenomic samples sufficient to allow species identification.

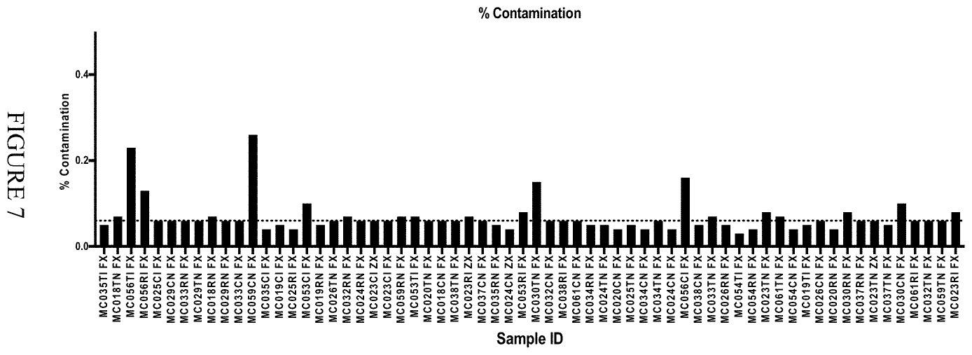

In addition to generating sufficient total raw read counts to enable high-quality metagenomic analysis, the methods of the invention have effectively eliminated any eukaryotic DNA contamination (FIG. 7) within the metagenomic samples.

To asses for potential human, mouse and adaptor sequence DNA contamination rates amongst the 64 metagenomic samples, sequence trimming of the raw reads was performed using Trimmomatic v.0.38 to ensure that all technical sequencing defects were removed and guarantee that only clean, raw reads remained. The raw reads were then mapped against the human reference genome (hg19), mouse reference genome (mm10) and adapter sequence (adapters) reference sequences using bowtie2 to assess for the presence of contaminating reads. These methods confirmed the effective elimination of eukaryotic reads from the metagenomic samples, with a maximum potential eukaryotic contamination level of 0.26%, a minimum of 0.03% and a median of 0.06% (FIG. 7).

Overall, using these methods, the inventors have shown that combining broad-spectrum anaerobic culturing techniques with metagenomic sequencing is able to effectively generate sufficient total raw read counts (FIG. 6) from metagenomic sequencing, while effectively eliminating any potential eukaryotic contamination amongst the samples (FIG. 7).

### Example 5—Metagenomic Sequencing of Cultured Bacterial Communities from Lung Bronchoalveolar Lavage Fluid Samples

### Method

Nasopharyngeal swab (NPS)/oropharyngeal swab (OPS) and non-bronchoscopic lavages (BALs) were obtained using NPS/OPS (BD ESwab™ Collection and Transport System; Franklin Lakes, United States) and lavage kit (Unomedical Tracheal Suction set; ConvaTec Limited, Flintshire, United Kingdom). The NPS/OPS contained 1 ml of saline which the swab was submerged in. The sputum trap provided in the lavage kit was connected to the patients existing in-line suction and a small volume of lavage fluid (sterile NaCl 0.9%) was added into the endotracheal tube, passing down into the lung. The lavage volume was calculated according to the patient's weight at the time of sampling (Table 2).

TABLE 2
BAL volumes

| Baby/Child | | | |
|---|---|---|---|
| Weight | | | |
| Range (at time | | BAL | |
| of sampling) | | Volume | |
| 0.5-1 | kg | 0.7 | ml |
| >1 | kg | 0.7-1.0 | ml |
| >1.5 | kg | 1.0-1.25 | ml |
| >2 | kg | 1.25-1.5 | ml |
| >3 | kg | 1.5 | ml |

The resulting samples were plated on Brain Heart Infusion Media (BHI) (Amyl Media), Anaerobic agar (ANAE) (Thermo-Fisher Scientific), Chocolate agar (CHOC), Fastidious anaerobe agar (FAA) (Thermo-Fisher Scientific), Fastidious anaerobe agar with 5% defibrinated horse blood (FAHB) (Thermo-Fisher Scientific), yeast-extract-casitone-fatty acid (YCFA) and Wilkin's-Chalgren anaerobe agar (WILK) (Amyl Media) in each of three (anaerobic (10% CO2, 10% H2, 80 N2), microaerophilic (2.5 L CampyGen gas pack (Oxoid; Basingstoke, Hampshire, United Kingdom)) and aerobic) atmospheric conditions.

After incubation, 1.5 ml of PBS was added to the agar plate and a spreader was used to disrupt samples prior to collection. Resulting samples (minimum 700 ul) were transferred to an Eppendorf tube and subjected to metagenomic sequencing.

Genomic DNA (gDNA) samples were diluted to ˜1 ng/μl and the DNA concentration was determined using the Qubit dsDNA HS Assay Kit (Thermo-Fisher Scientific) as per the manufacturer's protocol. Library construction was performed using the Illumina Nextera XT DNA Library Prep Kit (Illumina; San Diego, Calif., United States) at the MHTP Medical Genomics Facility (Clayton, Victoria, Australia). The kit required a DNA input of 1 ng, calculated using the diluted samples. The kit included transposomes that fragmented the gDNA and added adaptors i7, i5, P5 and P7, required for library pooling and sequencing. The samples underwent 12 cycles of amplification. The final libraries were quality checked by Qubit and library size was checked using the Agilent Bioanalyzer (Agilent; Santa Clara, Calif., United States). Final libraries were pooled using Qubit and Bioanalyzer and sent to the AGRF to be sequenced on an Illumina NovaSeq Instrument (Illumina) with 110 bp paired-end (PE) sequencing. Sequences were obtained as FastQ files containing paired reads and bioinformatically analysed.

### Results

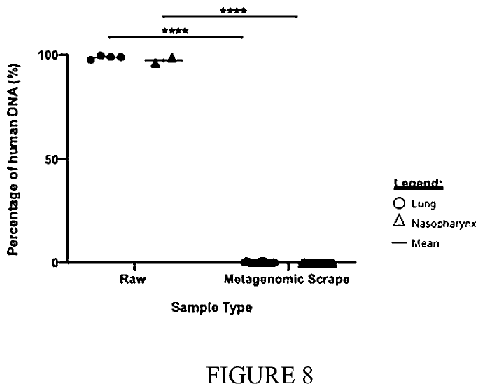

This data demonstrates applicability of this method for depletion of human DNA from lung and nasopharynx samples and the applicability of multiple bacterial culture media types (FIG. 8).

The present application claims priority from AU 2020900450 filed 18 Feb. 2020, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Almeida et al. (2019) Nature 568:499-504.
Awany et al. (2109) Front Genet. 9:637.
Browne et al. (2016) Nature 533:543-546.
Forster et al. (2019) Nat. Biotechnol. 37:186-192.
Kumar et al. (2017) Virus Res. 239:172-179.
Quince et al. (2017) Nat Biotechnol. 35:833-844.
Sharpton (2014) Front Plant Sci. 5:209.
Wang et al. (2015) World J Gastroenterol 21:803-813.
Wood and Salzberg (2014) Genome Biology 15:R46.

1. A method of identifying microorganisms of the microbiome of a region of a subject, the method comprising;
i) obtaining a metagenomic sample derived from the region depleted of nucleic acids from the subject,
ii) conducting metagenomic sequencing of nucleic acids in the depleted metagenomic sample from step i), and
iii) analysing the results of the metagenomic sequencing to identify microorganisms present in the microbiome in the region of the subject.

2. The method of claim 1, wherein step i) comprises one or more or all of:
1) culturing in vitro microorganisms from a sample of the microbiome from the region of the subject,
2) hybridizing a probe to DNA of the subject in the metagenomic sample, and depleting the sample of DNA bound to the probe, and
3) hybridizing a probe to DNA of microorganisms expected to be present in the metagenomic sample, and selecting DNA bound to the probe.

3. The method of claim 1 or claim 2, wherein step i) comprises
a) culturing in vitro microorganisms from a sample of the microbiome from the region of the subject, and
b) obtaining a metagenomic sample from the cultured microorganisms.

4. A method of identifying microorganisms of the microbiome of a region of a subject, the method comprising;
i) culturing in vitro microorganisms from a sample of the microbiome from the region of the subject,
ii) obtaining a metagenomic sample from the cultured microorganisms,
iii) conducting metagenomic sequencing of nucleic acids in the metagenomic sample from step ii), and
iv) analysing the results of the metagenomic sequencing to identify microorganisms present in the microbiome in the region of the subject.

5. The method according to any one of claims 2 to 4, wherein the sample is cultured under anaerobic conditions.

6. The method according to any one of claims 2 to 4, wherein the sample is cultured under aerobic conditions.

7. The method according to any one of claims 2 to 4, wherein the sample is cultured under microaerophilic conditions.

8. The method according to any one of claims 2 to 7, wherein the microorganisms are cultured on yeast-extract-casitone-fatty acid (YCFA) agar.

9. The method according to any one of claims 2 to 8, wherein the microorganisms are cultured at about 37° C.

10. The method according to any one of claims 1 to 9, wherein the subject is an animal or a plant.

11. The method of claim 10, wherein the animal is a mammal.

12. The method of claim 11, wherein the mammal is a human.

13. The method of claim 10 or claim 11, wherein the region is selected from a region of the gastrointestinal system, the respiratory system, the female reproductive system, the bladder or the skin.

14. The method of claim 13, wherein the region of the gastrointestinal system is a region within the stomach, small intestine, large intestine, caecum or rectum.

15. The method of claim 14, wherein the region is the terminal ileum of the small intestine.

16. The method of claim 13, wherein the region of the respiratory system is a region within the lung.

17. The method of claim 19, wherein the region of the female reproductive system is the vaginal region.

18. The method according to any one of claims 1 to 17, wherein the sample is from a region of the subject with a phenotype of interest.

19. The method of claim 18, wherein the phenotype of interest is a diseased state.

20. The method of claim 19, wherein the region is inflamed.

21. The method according to any one of claims 1 to 20, wherein the microorganisms of the microbiome comprise bacteria, fungus, protozoa, viruses, or any combination thereof.

22. The method of claim 21, wherein the microorganisms of the microbiome at least comprise bacteria.

23. The method of claim 21 or claim 22, wherein the viruses include bacteriophages.

24. The method according to any one of claims 1 to 23, wherein step iv) comprises comparing the sequences identified in step iii) to a database comprising microbial sequences.

25. A method of identifying a microorganism which may be associated with a phenotype of interest, the method comprising
i) performing the method according to any one of claims 1 to 24, wherein the sample is from a region of the subject with a phenotype of interest,
ii) comparing the microorganisms identified in step i) with those present in the same region of a subject that does not have the phenotype of interest,
wherein microorganisms identified in step i), but which are not present at the same level in the same region of a subject that does not have the phenotype of interest, may be associated with the phenotype of interest.

26. A method of identifying live microorganisms present in a food, drink or probiotic composition, the method comprising;
i) obtaining a metagenomic sample derived from the food, drink or probiotic composition depleted of nucleic acids from a source other than the live microorganisms,
ii) conducting metagenomic sequencing of nucleic acids in the depleted metagenomic sample from step i), and
iii) analysing the results of the metagenomic sequencing to identify live microorganisms present in the food, drink or probiotic composition.

27. The method of claim 26, wherein step i) comprises one or more or all of:
1) culturing in vitro microorganisms from the food, drink or probiotic composition,
2) hybridizing a probe to DNA of the subject in the metagenomic sample, and depleting the sample of DNA bound to the probe, and
3) hybridizing a probe to DNA of microorganisms expected to be present in the metagenomic sample, and selecting DNA bound to the probe.

28. The method of claim 26 or claim 27, wherein step i) comprises
a) culturing in vitro microorganisms from the food, drink or probiotic composition, and
b) obtaining a metagenomic sample from the cultured microorganisms.

29. A method of identifying live microorganisms present in a food, drink or probiotic composition, the method comprising;
i) culturing in vitro microorganisms from the food, drink or probiotic composition,
ii) obtaining a metagenomic sample from the cultured microorganisms,
iii) conducting metagenomic sequencing of nucleic acids in the metagenomic sample from step ii), and
iv) analysing the results of the metagenomic sequencing to identify live microorganisms present in the food, drink or probiotic composition.
